(19) United States
(12) Patent Application Publication
(10) Pub. No.: US 2001/0034325 A1
(43) Pub. Date: Oct. 25, 2001

(54) Dietary modulators of gamma glutamyl transpeptidase

(76) Inventor: Vladimir I. Slesarev, Coeur d' Alene, ID (US)

Correspondence Address: Vladimir Slesarev, 201 North 17th St., Coeur d' Alene, ID, 83814, US

(21) Appl. No.: 09/765,734

(22) Filed: Jan. 18, 2001

Related U.S. Application Data
(63) Continuation-in-part of application No. 09/520,131, filed on Mar. 7, 2000, now abandoned.

(51) Int. Cl. A61K 38/16

(52) U.S. Cl. 514/8

(57) ABSTRACT

The invention relates to the new compositions comprising probiotic glucoproteins and isolated soy proteins for lowering upregulated gamma glutamyl transpeptidase humans or domestic animals. Their synergetic effects with N-acetyl glucosamine is disclosed.

### RELATED APPLICATIONS This appcation continues in part from pending application Ser. No. 09/520,131 filed Mar. 7, 2000.

### BACKGROUND

Adenosine triphosphate (ATP) independent enzyme, gamma glutamyl transpeptidase (GGTP) has been postulated to be a part of an amino acid uptake system. This membrane bound ectoenzyme is responsible for hydrolysis of extracellular glutathione (GSH), and is required for the maintenance of normal intracellular GSH levels. GGTP cleaves extracellular GSH, thus providing cells with cysteine, dipeptides cystein-glycine, and gammaglutamyl-glycine.

Elevation of GGTP activity leads to the deficiency of the antioxidant glutathione and appears to be connected with several cardiovascular, neurological, and oncological diseases. GGTP levels are strongly associated with all-cause mortality, largely due to a significant increase in deaths from ischemic heart disease and other non-cardiovascular disease (Wannamathee G., et al, Am. J. Epidemiol., 1995,). In this prospective study of 7,613 British men elevation of GGTP was significantly associated with preexisting ischemic heart disease, systolic and diastolic blood pressure, total and high density lipoprotein cholesterol, heart rate, blood glucose, diabetes mellitus, alcohol intake, body mass index, smoking, and negatively associated with physical activity and lung function. Increased risk of ischemic heart disease mortality was noticed particularly in patients with previous myocardial infarction. These epidemiological data have been confirmed later in detailed pathogenetic study, which provided mechanistic clues to the epidemiological evidence for a possible correlation between persistent elevation of GGTP and the risk of fatal reinfarction in patients with ischemic heart disease (Paolicchi A., et al. J. Investig. Med, 1999; 47 (3):151-160.). Fe (III) reduction and low density lipoprotein (LDL) oxidation were increased remarkably after addition of purified GGTP. GSH-dependent LDL oxidation was similarly promoted by GGTP associated with the plasma membrane of human monoblastoid cells, and this process required iron traces that can be found in advanced or late stage atheromas. Elevation of GGTP, aspartate transaminase, and bilirubin have prognostic importance in chronic heart failure (Batin P., et al., Eur. Heart J., 1995; 16:1613-8). Multiple system organ failure after cardiac surgery in children is also associated with increased GGTP activity. (Heying R., et al., 1999; 88:1238-43).

GGTP has been shown to be involved in the depletion of glutathione in the ischemic rat striatum. (Orwar O, et al. J.Neurochem., 1994; 63:1371-6). Increased GGTP activity was correlated with neurological impairment in 42 females with an ischemic cerebral infarction (CI). GGTP serum level increases after CI as consequence of brain damage and this increment could be considered as a clinical and prognostic unfavorable index of the disease (D'Erasmo E., et al., Biomed. Pharmacother, 1993; 47:89-92).

It is interesting that a very early event in the pathogenesis of Parkinson's disease is a massive loss of GSH in the substantia nigra. The fact that active metabolite of the dopaminergic neurotoxin 1-methyl-4 phenyl-1,2,3,6-tetrahydropyridine causes the loss of nigrostriatal GSH without corresponding increases of glutathione disulfide suggests that the release and GGTP-mediated hydrolysis of GSH to glutamate, glycine, and cysteine may be important pathogenic stage of the degeneration of dopamine neurons (Han J., et al. J.Neurochem., 1999; 73:1683-95). There is also a significant correlation between the GGTP activity after nontraumatic intracranial hemorrhages and exposure to hepatotoxic drugs, particularly phenotoin (Meythaler J M., et al. Arch.Phys.Med.Rehabil., 1998, Jul;79:766-71). In addition, hepatotoxic anaesthetics halothane, enflurane, and even less toxic isoflurane and sevoflurane induce an elevation of serum GGTP in neurosurgical patients (Nishiyama T., et al. Can.J.Anaesth. 1998; 45: 753-6).

Furthermore, GGTP is directly contributing to oxidative damage of ischemic rat kidney. Ex vivo data supporting a net pro-oxidant effect of up-regulated GGTP during short-term ischemia have been obtained (Cutrin J C., et al. Kidney Int. 2000; 57: 526-533.).

The peripheral blood of patients with rheumatoid arthritis was found to have expanded numbers of T cells expressing levels of GGTP up to 10-fold higher than controls. GGTP expression was up-regulated on peripheral blood T cells following activation in vitro by either superantigen, or IL-15, a stimulatory cytokine synthesized in rheumatoid synovium. (Karp D R., et al., Int.Immunol., 1999; 11:1791-800). GGTP activity was also elevated in dystrophic skeletal muscle hamsters and mice. (Neerunjun J S, and Dubowitz V., J.Neurol. Sci. 1977;33:335-40).

Increased GGTP activity appears especially harmful for cancer patients. Significant number cancer patients are undernourished at presentation and, despite dietetic support, endure considerable nutritional problems during radiotherapy. GGTP positive correlation with percentage weight loss during radiotherapy was found in patients with early laryngeal carcinoma. The biochemical markers, haemoglobin and albumin, were normal in the great majority at presentation. (Collins M M, et al Ann.R.Coll.Surg.Engl., 1999; 81:376-).

Elevated GGTP levels and intracellular glutathione in tumor cells have been correlated with resistance to several classes of chemotherapeutic drugs. Moreover, GGTP positive tumors grew at more than twice the rate of the GGTP-negative tumors. GGTP-positive tumors were significantly more resistant to the toxicity of cisplatin than the GGT-negative tumors. On the other hand, expression of GGTP is required for the nephrotoxicity of cisplatin. (Hanigan M H, et al. Carcinogenesis 1999, 20:553-9). In addition, GGTP activity was significantly increased in bronchoalveolar lavage fluid throughout pneumotoxicity. (Day B J, et al., J. Pharmacol. Methods, 1990; 24:1-8). It leads to drastic depletion of total glutathione in in alveolar epithelial lining fluid of patients with Adult Respiratory Distress Syndrome (ARDS) (Bunnel E. and Pacht E., 1993;148:1174-8). Thus, GGTP elevation causes excessive catabolism pulmonary glutathione, a tripeptide that is able to react with and effectively neutralize oxidants, such as hydrogen peroxide and superoxide anion. This oxidant may react to form the very toxic hydroxyl radical (OH) in body fluids in the presence of a transitional iron catalyst via Haber-Weiss reaction. Because only traceable quantities of free iron are required for this catalytic enhancement radical production, the availability of iron is tightly regulated by ferritin, an intracellular iron storage protein, which converts ferrous (Fe.II⁺) iron to the ferric (Fe.III⁺) state. Ferritin also exists in the serum, and serum ferritin levels usually correlate with total body iron stores. Still, superoxide anion, acidosis, and nitric oxide can release iron from ferritin.

Few GGTP inhibitors were checked for therapeutic effects. Two of them, diallylsulfide (DAS) and indole-3-carbinol (I3C), are constituents of garlic and inhibited significantly GGT levels in a strong carcinogenic mitogen (12-o-tetradecanoyl phorbol-13-acetate) induced activity. Therefore, these dietary constituents seem to be strong modifiers of chemically induced carcinogenesis. Another GGTP inhibitor, acivicin, interferes with glutamine metabolism, “targets” activated cells of the immune system and thereby attenuates the process and prevents overt diabetes, without major disturbance of glutamine levels or generalized immunosuppression. (Misra M, et al. Can. J. Physiol. Pharmacol., 1996;74:163-72). This potent GGTP inhibitor was tested as anticancer drug (Maroun et al. Cancer Treat. Rep., 1984; Acivicin was also used as antiacne agent (Castelhano, et al. U.S. Pat. No. 4,929,630 ).

All previously reported inhibitors of transglutaminase (covalent inactivators, alternative substrate inhibitors, and active site direct inhibitors) are effective only in high concentration, i.e. 10 sup.-3M or higher. Moreover, cystamine, active site direct inhibitor, and isocyanates, covalent inhibitors, lack specificity for GGTP.

Therefore, a need exists for safe, specific, and potent GGTP inhibitor.

### BRIEF SUMMARY OF INVENTION

The present invention is based on the discovery that probiotic glucoproteins inhibit elevated GGTP activity in humans. Consequently, in one aspect the invention provides new glucoprotein compositions, which demonstrate inhibition of GGTP activity and are useful for treating oxidative stress after ischemic reperfusion injury, atherosclerosis, heart attack, cerebral infarction, and chronic heart failure. Applicant also proposes glucoprotein compositions which enhance the modulation of GGTP expression by specifically eliminating immunogenic impurities from it, thus decreasing the compositions ability to stimulate macrophages mediated TNF alpha production and cytotoxicity.

Another aspect of the present invention is to provide a novel medical food consisting of the probiotic glucoproteins as free radical scavengers or radioprotective modifiers. Such medical food may be used to reduce GGTP associated malignancy and damage caused by radiation therapy and chemotherapy. Specifically, the present food may be recommended for those patients who suffer from common postchemotherapy toxicity such as leukocytopenia, thrombocytopenia, and high bilirubin with elevated liver enzymes. Further, the present invention provides nutrition for reducing cancer cachexia and muscle dystrophia.

In a related aspect, the present invention provides a food useful for treating patients suffering from hepatotoxicity caused by chemicals, anesthetics, drugs, and alcohol. Glucoprotein fortified food and drink may be especially beneficial for people with concurrent liver cirrhosis, thus preventing severe fatigue and brain damage caused by ammonia. Furthermore, presented invention provides the food for metabolic detoxications of the cancerogenic chemicals and mutagens.

Another aspect of the present invention includes nutritional methods of GGTP inhibition in patients with diabetes mellitus and rheumatoid arthritis. Therapeutic effect is based on newly discovered phenomena of the inhibitory effects of probiotic peptidoglycans over glutamine metabolism in immunocompetent cells. This inhibition targets only activated cells (with significant GGTP expression) of the immune system, and thereby attenuates the autoimmune process and prevents rheumatoid arthritis and overt diabetes without major disturbance of glutamine level or generalized immunosuppression.

Still another aspect of the present invention includes dietary methods of inhibiting dermal and eye GGTP, thereby reducing clinical symptoms of psoriasis and cataracts.

While another aspect of this invention is to provide a method to increase the extracellular glutathione levels in the lung of the patients with pulmonary diseases such as adult respiratory distress syndrome, fibrosis, cardiogenic lung edema, and lung cancer.

Yet another aspect of this invention comprises oral administration of the probiotic glucoproteins to decrease the level of serum iron in patients who are under oxidative stress. Examples of such conditions are sepsis, burn shock, pancreatitis, tumor lysis syndrome, hemotransfusion, and trauma. Probiotic glucoproteins are especially useful for reducing free Fe 2+ ion, thus preventing formation toxic free radical OH. A daily glucoproteins dosage in the range of from 10 mg to 800 mg may be found to be acceptable for ferrous iron reduction, with optimal range of 40-100 mg per day.

Still, another aspect of this invention coveres newly discovered phenomena of the inhibitory effects of soy proteins over elevated gammaglutamyl traspeptidase. Moreover, retained natural level of isolavones causes the synergetic effects with probiotic glucoproteins. The patients with elevated GGTP caused by hepatitis C, hepatocellular carcinoma, and Parkinson disease will benefit from this compositions. A daily dose of 50-75 g of isolated soy proteins with at least 0.1 weight percent of the retained isolavones is preferable surving quantity.

### BRIEF DESCRIPTION OF THE DRAWINGS

For further details, reference is made to the discussion which follows, in light of the accompanying drawings, wherein:

FIG. 1 illustrates the percentage change of the GGTP activity in cancer patients.

FIG. 2 demonstrates the changes in white blood cell counts in the same patients.

FIG. 3 presents the changes in thrombocytes counts.

FIG. 4. shows the effect on the serum bilirubin in cancer patients.

FIG. 5 demonstrates total iron changes after feeding with probiotic glucoproteins.

### DETAILED DESCRIPTION OF THE INVENTION

The present invention relates to dietary inhibition of GGTP and food and drinks, containing, as effective component, glucoproteins extracted from Gram positive bacteria. This invention also provides medical food for specific inhibition of elevated activity of GGTP, which may be administered orally to humans in single dose as small as 1 mg/kg. The dosage of 2-20 mg/kg may be preferable. Inhibatory effects over GGTP may be enhanced by isolated soy proteins. For the safety reasons, glucoprotein complexes from lactic acid bacteria such as Lactobacillus or Bifidum may be preferable.

Disaccharide peptides are the part of the basic unit of the glucoproteins-peptidoglycans. In the cell wall they are bound to teicholic acid and polisaccharide by a phosphate diester band. Basic unit was described by Sawasada et al (U.S. Pat. No. 5,234,904, 1993). However, under certain preparation conditions, two aminosugars (N-acetyl-glucosamine) are linked to muramic acid. This bond remains basically intact after lysozyme hydrolysis.

In parallel, a great deal of endotoxicity is caused by peptodoglycans derived from gram-positive bacteria. Its peptidoglyean is able to induce leukopenia and thrombocytopenia (Verhoef J. and Kalter E., Prog.Clin.Biol.Res. 1985; 189:101-113). Moreover, peptidoglycans and lipoteichoic acid can cause the induction of nitric oxide (NO) formation, shock, and organ failure in the rat (Kengatharan K, et al. J.Exp.Med., 1998;20:305-15). Short fragment, ,N-acetyl-glucosamine-muramyl dipeptide (GMDP), is responsible for synergizing with lipoteicholic acid thereby causing septic shock during gramm positive infection. Orally administered peptidoglycans enhance leukopenia by stimulating of the phagocytosis of splenetic neutrophils from mice. (Sasaki T., et al. J.Vet.Med.Sci. 1996, 58:85-6). In addition, peptidoglycans, well known tumor necrosis factor (TNF) alpha stimulators, promote rheumatoid arthritis inflammation.

It is well known fact that low molecular weight peptidoglycans, MDP (492 D) and GMDP (695 D), are mainly responsible for immunogenic effects. They are potent stimulators of TNF alpha production, which may be detrimental in patients with autoimmune conditions such as rheumatoid arthritis. Excessive level TNF alpha production could be extremely dangerous for patients with ARDS, stroke, and ischemic heart disease, who already have high preexisting production of TNF alpha. Moreover, combination of MDP and TNF alpha can cause proinflammatory effects, thus exaggerating chronic viral and bacterial infection. The present invention has been completed on the basis of findings that inhibition over GGTP activity caused by differences in the molecular weight of the glucoproteins of the gramm positive bacteria. The glucoproteins having molecular weight less than 30000 D obtained by lysozyme hydrolysis of gramm positive bacteria exhibit newly discovered antioxidant and detoxification effects. In parallel, undesirable proinflammatory and immunogenic properties were avoided by eliminating low molecular weight peptidoglycans, MDP and GMDP. It provides exceptional safety and improved tolerance in people with autoimmune conditions. In addition, achieved GGTP inhibition leads to the repletion of glutathione, well-known TNF alpha inhibitor.

The present invention provides an oral inhibitor of GGTP substantially free of low molecular weight peptidoglycans MDP and GMDP. The glucoprotein fraction of molecular weight of not higher than 30,000 D and not less than 800 D in the cell wall of gramm positive bacteria may be purified by a known method of molecular weight fractionation of proteins or by ultrafiltration. The presented glucoproteins for GGTP inhibition with concurrent inhibition of immunogenicity are significantly different from previous inventions related to probiotic peptidoglycans. All of them have presented probiotic peptidoglycans as the immunostimulators. Immunostimulatory properties were reported by Link and Pahud (U.S. Pat. No. 5,185,321, 1993) and by Yamazaki et al,(EP99104209, 1999). Moreover, Yamazaki et al. taught to increase immunogenicity by purifying low molecular weight fraction of 500-4000 peptidoglycans with increased percentage of very low molecular weight of 500 D peptidoglycans. Their main objective was to increase production of TNF alpha to the level comparable with the stimulation by muramyl dipeptide (MDP).

On the other hand, it has been currently reported that polysacharide peptidoglycans complexes isolated from probiotics exert antihypertention effects (U.S. Pat. No. 5,234,904, 1993). The The same effects have been noticed on the subjects who consumed isolated soy proteins. These proteins have been shown to reduce level of LDL cholesterol and total cholesterol, as well as to ameliorate symptoms of diabetes and astma (Hoie L. PCT/IB99/01992, 1999, PCT/IB99/01997).

The glucoprotein inhibitors of GGTP of the present invention have the following excellent features;

1. They are antioxidants and free radical scavengers originated from lactic acid bacteria, which are used in the production of yogurt and fermented milk food and drinks.

2. They are the substances from natural origin which can exert antioxidant effects at a dose much less than the known antioxidants produced by plants, sea weeds and microorganisms.

3. Since they are water soluble, they can be readily prepared in appropriate formulations. Latest feature is a real benefit for parenteral administration.

4. In composition with isolated soy proteins their effects over upregulated GGTP can be enhanced. Such food compositions also provide well-balanced daily source of the amino acids.

As stated above, GGTP inhibition leads to the preservation of extracellular glutathione powerful antioxidant with remarkable detoxification properties. In particular, the invention has application in alcohol detoxification, anesthetic recovery and in recovery or withdrawal from hypnotics, narcotics, sedatives or other drugs, especially in case of abuse. Treatment of withdrawal is a particular area where the invention has applicability. The invention may have application in the prevention, treatment or management of toxicity caused directly or indirectly by one of the following compounds:

anesthetics, including: local anesthetics (such as cocaine, procaine, lidocaine, tetracaine, mepivacaine, bupivacaine and etidocaine, chlorprocaine), inhalational anaesthetics (such as methoxyflurane, halothane, enflurane, isoflurane and nitrous oxide); intravenous anesthetics (etomidate, benzodiazepines, and barbiturates);

opiods, including: heroin and morphine related opioids (such as hydromorphone, oxymorphone, levorphanol, codein, hydrocodon, oxycodone, nalorphine, naloxone, naltrexone, buprenorphine, butorphanol and nalbuphine);

sedatives and hypnotics, including barbiturates and benzodiazepines;

other drugs subjects to abuse, including cocaine and related drugs; nicotine and tobacco; psychedelic drugs, which are hallucinogenics and/or psychotomimetics and/or psychotogenics;

ethanol and its metabolites.

The invention has applications in dealing with endogenous created toxins. Acetaldehyde, the primary metabolite of ethanol, is an example. Probiotic glucoproteins are useful for dietary management of the patients who accumulated endotoxins as a result of disease.

One endogenous toxin, which can often cause the problems, is bilirubin. High levels are known to results in jaundice, particularly in babies and patients with advanced hepatic metastases. Reduced liver function is also a characteristic feature of geriatric patients. In addition, in cancer patients, levels of drug such as analgetics and chemotherapeutics tend to build up in the body and this can lead to severe side effects. Yet, another endotoxins, free radicals, are generated during radiation and chemotherapy.

However, the applicant has been able to demonstrate that the level of metabolites such as bilirubin and iron in the blood of patients could be significantly reduced when patients are fed with probiotic glucoproteins.

Because of their exceptional inhibitory activity over GGTP, glucoproteins are extremely valuable food for combating leukocytopenia and thrombocytopenia, which are common toxic side effects of radiation and chemotherapy in cancer patients. Precisely how leukocytopenia and thrombocytopenia are treated and prevented remains to be shown. Though, it is well established fact that the inhibitors of glutathione transferase, such as sulfasalazine, an agent used to treat ulceratus colitis, prevents myelosuppresion in cancer patients treated with melphalan (Gupta V., et al. Cancer Chemother. Pharmacol. 1995;36:13-9). Glutathione's role as powerful antioxidant capable of preventing postchemotherapy leukopenia is well documented (Di Re F, et al., Cancer Chemother.Pharmacol., 1990;25:355-60). Importance of free radical scavengers in preventing neutropenia was also demonstrated (Linday L A, et al., J. Clin. Psychopharmacol., 1995;15:353-60). From the above it can be seen that the invention also relates to a method for the prevention, treatment and management of toxicity mediated by oxidative stress and free radical attacks. In addition, the invention relates to nutrition for reducing damage after ischemic/reperfusion injury, stroke, heart attack, ARDS, and Parkinson disease. Feeding with probiotic glucoproteins is particularly effective in those patients who have increased risk of secondary heart attack and stroke. Patients with severe chronic heart failure will significantly benefit from proposed medical food by improving their cardiac output and reducing congestive edema.

Because of this specific GGTP inhibition, probiotic glucoproteins are valuable for all conditions with upregulated GGTP activity. It seems that they are extremely useful for treating patients with ovarian and hepatocellular carcinoma, prostate cancer, pancreas and breast cancer.

Yet, among another indications are diabetes and autoimmune diseases such as rheumatoid arthritis. The present inventor has carried out intensive research in order to develop safer agents, which can exhibit profound desensitizing effect without significant immunosupression. Consequently, he has found out that the decreasing percentage of low molecular weight peptidoglycans by ultrafiltration of ingredients less than 1000 D and higher than 30000 D creates glucoproteins composition which does not stimulate TNF alpha production, thereby is absolutely safe for people with rheumatoid arthritis. The present invention provides a medical food, which can desensitize T lymphocytes without any immunosuppression.

The method for preparing medical food according to the present invention will now be explained. The glucoprotein compositions to be used in the present invention may be obtained from a variety of Gram positive bacteria following known methods. The amino acid sequence and sugar composition of the complexes will be defined by a species of lactic acid bacterium. All of the glucoprotein compositions obtained from various Lactobacillus (L.) strains, including L.acidophilus, L.casei, L.bifermentans, L.reuteri, L.alimentarius, L.helveticus, L.brevis, L.collinoides, L.coryneformis, L.crispatus, L.curuvatus, L.delbrueckii, Ljensenii, L.lactis, L.salivarus, L.murinus, L.Bulgaricus, L.Plantarum, etc.

Each lactobacillus is cultured via the culture conditions suitable for the microbiological properties of the species, to collect the cultured bacterial cells. These may be cultured in the culture medium routinely used for lactobacillus, for example, Rogosa medium, but complex medium using soy protein broth or distiller's soluble, etc. as nitrogen source may be also used. Peptones, yeast extracts, and glucose are most preferable ingredients of culture medium. The fermentation methods may follow the routine methods for lactobacilli. Routine methods for bacteria degradation such as ultrasound, temperature (hot water), and enzyme hydrolysis may be employed. Lysozyme hydrolysis is preferable one.

Routine methods for purifications of glucoprotein complexes can be employed. More specifically, hydrolysate obtained by aforementioned methods, is applied to anion-exchange column to remove lysozyme and high-molecular nuclear acids. Further, protease and nuclease can be used for degradation of the remaining proteins and nuclear acids, respectively. Hydrophobic chromatography may be used to remove enzymes by passing them through a column with resin. Glucoprotein composition may be fractioned by gel chromatography.

Yet, ultrafiltration by using 400 D, 1000 D, and 30000 D membranes are considered most preferable method for purifications of the glucoprotein compositions. More specifically, 30000 D polyethersulfone membrane may be used to filtrate nuclear acids and high molecular weight proteins (including lysozyme) from aforementioned lysozyme hydrolysite. Then, 1100 D regenerated cellulose membrane may be employed to eliminate low molecular weight (immunogenic) peptidoglycans from glucoprotein composition. This high molecular fraction is indicated for GGTP inhibition in patients with autoimmune diseases, when immunogenic, proinflammatory peptidoglycans with low molecular weight may cause severe side effects. Similarly, fractions, which compose of different percentage of low molecular weight glucopeptides, can be obtained by using 350 D reverse osmosis membrane and 10000 D or 30000 D polyethersulfone membranes. 350 D or 800 D membranes are used for filtrating low molecular pyrogenic muramyl peptides and glucomuramyl peptides, respectively, as well as salts and acetic acid. Employing 100000 D or 30000D membranes can regulate percentage of high molecular weight glucoproteins. Fractions obtained by aforementioned ultrafiltration are especially effective for inhibition of serum GGTP in patients with cardiovascular disorders, leukocytopenia, and ovarian carcinoma.

A prefered glucoprotein compositions can be obtained by mixing with isolated soy proteins and N-acetyl-glucosamine. The amount of the probiotic glucoproteins of the total weight of a composition on dry basis is preferably more than 10 weight percent.

Prefered amounts of N-acetyl-glucosamine as weight percent shall be in the range of from about 10 to 30 percent, for example such as 20 weight percent.

Accordingly, weight ratio of isolated soy proteins is preferably more 1.0, for example 1.15. Water prosseed soy proteins retaining a natural level of isoflavones are most preferable for mixing with probiotic glucoproteins.

Alternatively, the present invention provides a composition wherein probiotic and soy aminoacids serve as a daily source of protein.

The proposed composition can be served as a powder mixed with milk, orange juice or other beverage of choice.

### DESCRIPTION OF THE PREFERRED EMBODIMENTS

Specific production embodiments are presented hereinafter.

### EXAMPLE 1

### Isolation of Glucopeptides from Lactobacillus Reuteri

1. Biomass Preparation

Fermentation of Lactobacillus reuteri was done in 300 L fermenter with working volume of 220 L. Liquid nutrient medium was composed of 10 g/L of yeast extract, 20 g/L glucose, 20 g/L pepton+beef extract, 2 g/L K2HPO4, 5 g/L CH₃COONa, 0,2 g/L MgSO₄, 0,05 g/L MnSO4, and 0,1 g/L Tween 80.

Fermenting was made anaerobic conditions at temperature 40+−3° C. Fermenting period was 22 hours. Biomass was separated from the suspension on centrifuge Sorvall 3B at 4000 r/min. Moisture biomass yield was 1,6 kg, dried one −440 g. Wet biomass was rinsed twice by 1 L of distilled water, then was suspended in 1 L of CH3COONa for 2-4 hours, and again was rinsed by water using centrifuge Servile-5B at 8000 r/min. Biomass was exposed for 1-2 hours in 1 L of CH COONa at 60-80° C. and rinsed by distilled water. Then, 50-70% ethanol was used to rinse the biomass until supernatant liquid becomes colorless. 96% ethanol was used to stabilize the biomass. This processing was needed to preserve the biomass at room temperature for following hydrolysis. Moisture biomass should be stored at −60° C.

2. Hydrolysis.

1,6 kg moist biomass was rinsed by distilled water and 0,5M CH₃COOH, then resespended in 2.5 L H₂O and kept at 90° C. for 20-30 min. After that, it was diluted in 10 L H₂O+70 g NaHCO₃ (to achieve _{P}H=6.0) and added 8 g of lyzosyme (Canadian Inovatech, Inc., Vancouver, Canada). Hydrolysis was done for 11.5 H in the shaking incubator at 54° C. Then, 110 ml CH₃COOH was added to achieve pH=4.0 and was centrifuged on Beckman J-6 g at 4000 rpm for 30 min. (12 L obtained solution has contained a lot of pigments).

3. First Ultrafiltration.

Cartridge with the membrane capable of retaining compounds with molecular weight less than 30 000 D and with S=0.09M² at speed 2.5 L/h (Millipore Corp, USA) were used. 1.8 L solution with retained nuclear acids, phospholipids, and lysozyme was wasted. 10 L was passed through column with micropore cationite in H-form in order to eliminate residual lysozyme and pigments.

4. Second ultrafiltration

Cartridge with Nanomax (Millipore Corp., USA) membrane filtrating compounds with molecular weight less than 500 D (muramylpeptides, salts, acetic and lactic acids) was used. Then, 1 L of retained glucoproteins was filtrated through 0.45 microns filter and freeze dried. Yield was 11 g high molecular weight glucoproteins.

### EXAMPLE 2

### GGTP Bition in Patient with Chronic Heart Failure

Patient C, 72 years old, white female, has presented for evaluation of her progress on antihypertensives and her history of congestive heart failure. She stated she was having fatigue and shortness of breath. She was on Captopril 50 mg t.id., Zaroxol 2.5 mg daily, Cordarone 200 mg daily, Digoxin 0.25 mg every other day, Coumadin 5 mg tablets ½ on even days and a full tablet on odd days, progesterone 100 mg daily, Tri-eastrogen 2.5 mg daily and thyroid 4 grains daily. History is only significant for hypothyroidism, hypertension, congestive heart failure and psoriasis.

Her laboratory tests revealed a nonfasting blood sugar of 172, GGTP elevated at 73 U/L, triglycerides elevated at 421, BUN 36, creatinine 1.4, globulins slightly elevated at 4.1 and serum ferritin 335 ng/ml. Her PT was 17.9. Her previous EKG revealed cardiac output reduced to 27%.

On exam her weight is 179 pounds. She is 5′1½″ tall, blood pressure 130/76, and pulse 72. She is a well developed, nourished and hydrated, moderately overweight, short-statured female, alert and oriented ×4. Her extremities reveal slight cyanosis and moderate +2 pitting edema. There are psoriatic patches on her lower extremities and trunk.

Impression:

1. Congestive heart failure, stable

2. Hypertension, controlled.

3. Psoriasis, stable

The plan was to obtain a digoxin level and chemistry panel. 40 mg daily of Glucoprotein extract of L.Bulgaricus was recommended as a medical food.

Two weeks later the patient is being seen after her follow-up echocardiogram and EKG. She states that she is feeling notably improved. She has no complaints of edema. Vital signs: She easily steps up to the exam table and down without any hesitation or shortness of breath. Weight is 180 pounds, blood pressure is 138/86, pulse is 68. Heart has a slightly irregular rhythm with no murmur. Normal S1 and S2 with no thrill or gallop. PMI is over the left apex. She has slight +1 pitting edema at the ankles bilaterally. Psoriasis patches were significantly reduced in comparison with previous examination. GGTP and triglycerades level is in normal range. There is remarkable two fold reduction reduction in ferritin concentration −166 ng/ml. Fasting sugar is 136 mg/dL. Her follow-up echocardiogram reveals a 53% ejection fraction with marked improvement compared to the previous one performed a month ago. Her Holter monitor confirms atrial fibrillation and premature ventricular contractions with a rare couplet. Electrocardiogram confirms a atrium fibrillation chronic. The impression is 1) resolving congestive heart failure; 2) hypertension, stable 3) resolving psoriasis.

### EXAMPLE 3

### GGTP Inhibition in Patients with Colorectal Cancer, Ovarian and Hepatocellular Carcinoma

Two females with ovarian carcinoma, two females with colorectal cancer, and one male with hepatocellular carcinoma were evaluated on GGTP inhibition by glucoprotein extract of L. acidophilus. All of them have liver or lung metastasis and received chemotherapy, which resulted in development of neutropenia, thrombocytopenia, lymphocytopenia, elevated direct bilirubin, GGTP and alanine transaminase. Females have developed severe ascites, which required fluid aspirations.

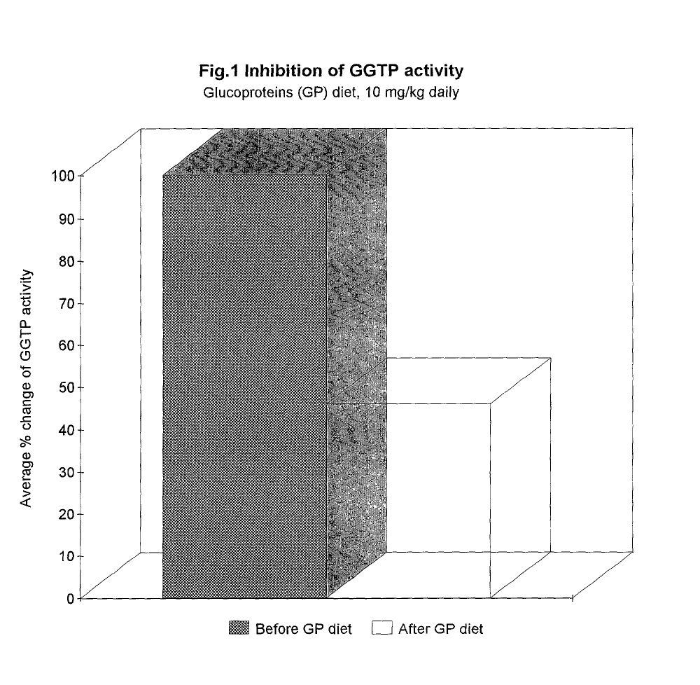

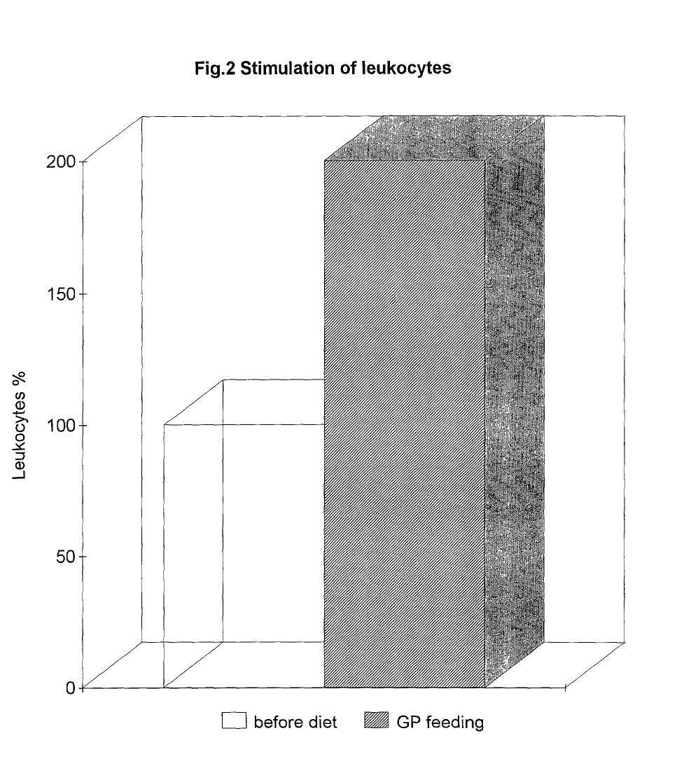

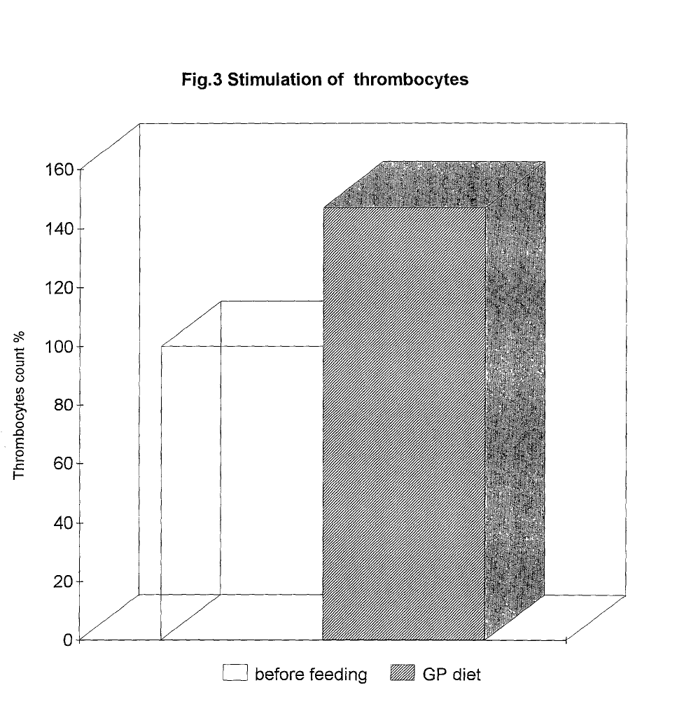

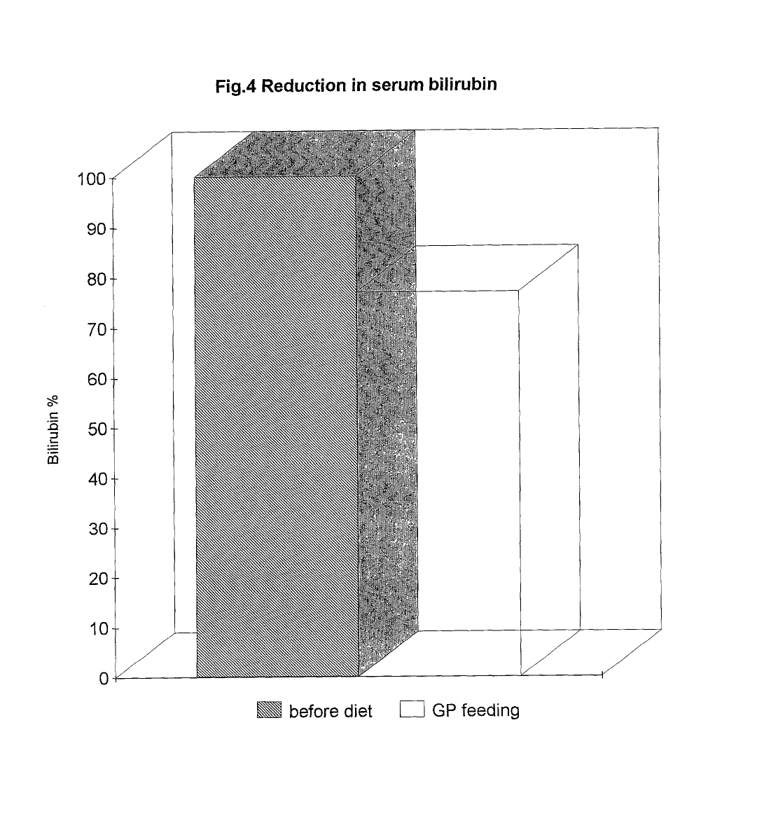

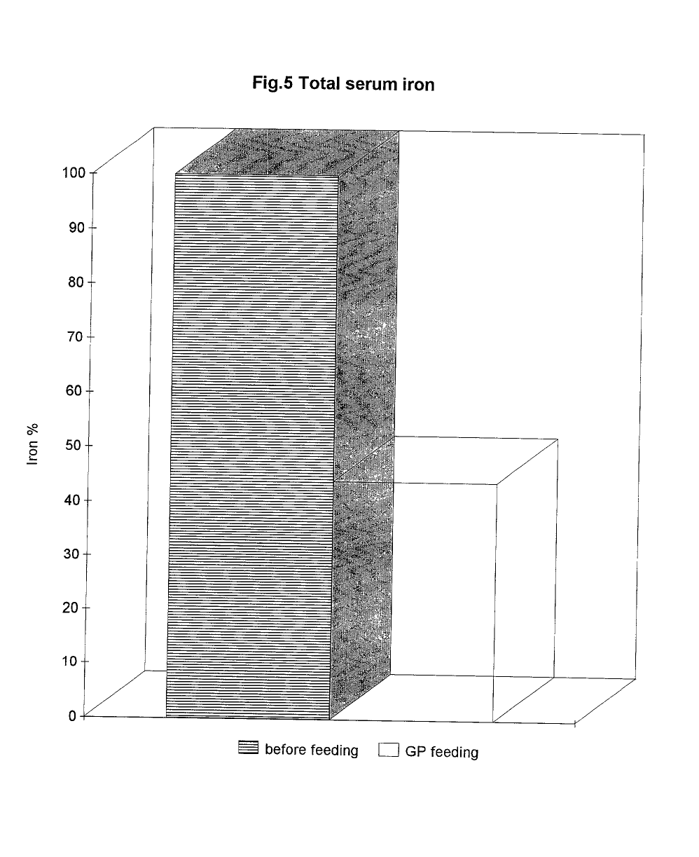

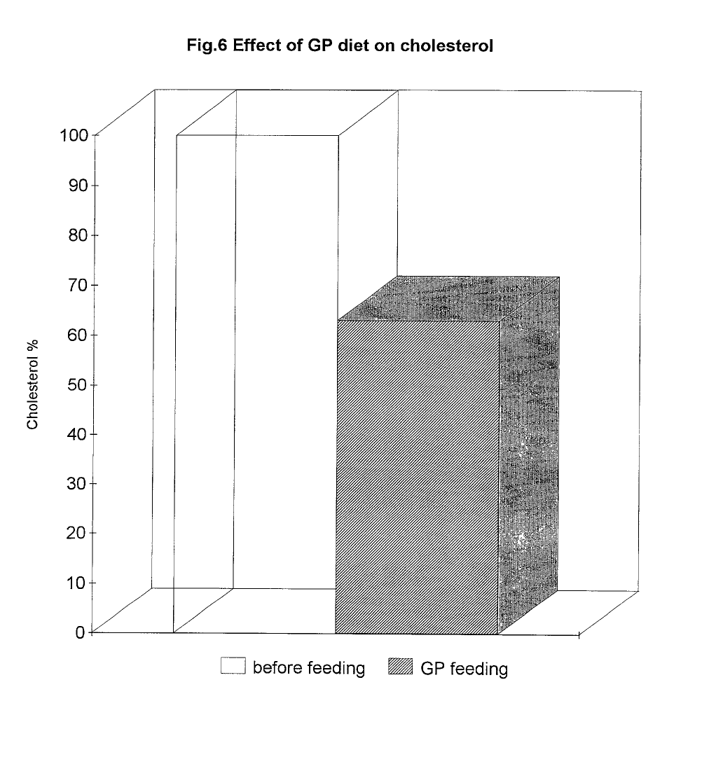

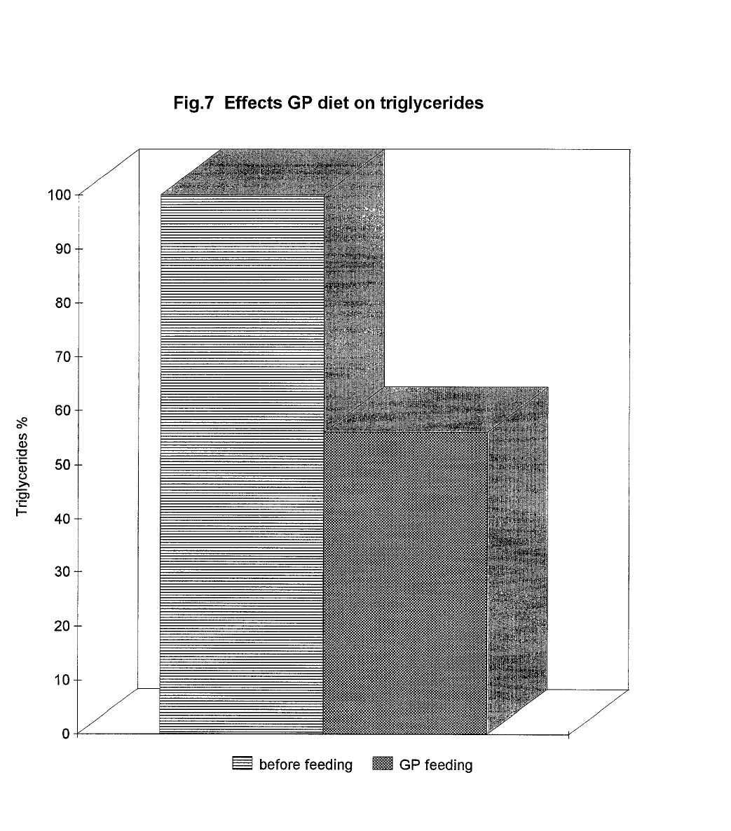

All patients were fed with probiotic glucoprotein for 3 weeks at dose of 5 mg/kg of body weight. Their general condition improved significantly. All patients noticed positive effect on fatigue and nausea. Effects on GGTP activity was evaluated as a percentage change of its level measured before feeding. FIG. 1 represents these changes. One can see 54% inhibition in patients with colorectal cancer and ovarian carcinoma. Yet, additional beneficial effects were noticed on ascites. Two women managed to get by without fluid aspirations, when they were on probiotic glucoprotein diet. FIG. 2 and FIG. 3 demonstrate the percentage changes of leukocytes and thrombocytes, respectively. There was two-fold increase in white blood cell count, as well as up to 47% increase in platelet count.

### EXAMPLE 4

### Probiotic Glucoprotein Diet for Patient with Diabetes

17 years old male was complaining of polyuria, polyphagia, and polydypsia over past month, as well as a upper respiratory viral infection one month previous to onset of the above symptoms. His family history was significant for insulin-dependent aunt on his father's side.

A physician who diagnosed Type I insulin-dependent juvenile-onset diabetes mellitus with a blood sugar of 972 mg/dL evaluated him. He was hospitalized and managed with insulin therapy, intravenous rehydration and nutritional guidance on diet and exercise. During his hospital course he was not at all ketonic. Subsequently, he was placed on 15 units of MPH and 5 regular in the morning and 10 MPH and 5 regular in the evening.

The patient was put on core diet program +80 mg of probiotic glucoprotein composition. Two months later, after being placed on a low fat, high complex carbohydrate diet, stressing supplementation, probiotic glucoproteins and exercise, he has been able to discontinue all his insulin and has maintained at this point, two months euglycemic.

### EXAMPLE 5

### Inhibition of Serum GGTP in the Patients with Hepatitis C

The primary objective of this study was defining the synergetic effects of the isolated probiotic and soy proteins on serum GGTP in patients with hepatitis C. Twenty-four patients with mean baseline serum GGTP level of 81 U/L were given 75 g of isolated soy proteins (Protein International, Inc. St. Louis, Mo.) and 10 g of glucoproteins extracted from L. Acidophillus (Neutraceutics, Inc. Redmond, Wash. and Chr. Hansen, Inc. Melwaukee, Wis.). All of them have consumed composition which preserved (according to Protein Technologies International, Inc.) natural level of daidzein, genistein, and glycitein varied from 150 mg to 225 mg. Seven patients were given placebo.

The preparation was taken as three daily liquid supplements for four weeks in the patient's regular diets. The mean reduction of GGTP in the treated group after four weeks was 27%, whereas in placebo group was no reduction in GGTP activity.

These clinical findings show that composition comprising isolated soy and probiotic proteins significantly inhibits elevated activity of serum GGTP and may improve prognosis in hepatitis C patients.

1. A medical food composition for feeding humans and domestically useful animals, which have a disease state, characterized by elevated gamma glutamyl transpeptidase activity comprising administering a therapeutically effective amount of probiotic glucoproteins.

2. A composition of claim 1 wherein said disease state is chronic heart failure

3. A medical food composition for modulating gamma glutamyl transpeptidase activity comprising at least 80 weight percent of isolated soy proteins, 20 weight percent of rhamnose, and 10 weight percent of the probiotic glucoproteins.

4. A composition of claim 1 wherein said disease is postchemotherapy leukocytopenia.

5. A composition of claim 1 wherein at least 50 weight percent carbohydrate moiety of the said glucoproteins is N-acetyl-glucosamine.

6. A composition according to claim 1 for lowering serum levels of GGTP in patients with acute respiratory distress syndrome.

7. A composition of claim 1 wherein said disease is Parkinson disease.

8. A composition of claim 1 wherein said disease is a brain stroke

9. A composition of claim 1 wherein said disease state is ischemic/reperfusion injury

10. A composition of claim 3 for lowering skin gamma glutamyl transpeptidase in the patients with psoriasis.

11. A composition of claim 1 for lowering eye gamma glutamyl transpeptidase in the subjects with cataract.

12. A composition of claim 1 wherein the disease state is acne.

13. A composition of claim 1 wherein said disease state is alcohol intoxication.

14. A composition of claim 1 wherein the disease state is anesthetics intoxication

15. A composition of claim 1 wherein medical food comprises glucoproteins with molecular weight in range of 400 D-10000 D.

16. A composition of claim 1 wherein medical food consists of glucoproteins with molecular weight in a range of 10000 D-30000 D.

17. A composition of claim 1 wherein the disease state is caused by diabetes.

18. A composition of claim 1 wherein the disease state is rheumatoid arthritis.

19. A composition of claim 1 wherein said disease state is postchemotherapy ascites.

20. A composition of claim 1 wherein said disease state is atherosclerosis.
